(19) United States
(12) Patent Application Publication
(10) Pub. No.: US 2015/0238347 A1
(43) Pub. Date: Aug. 27, 2015

(54) DENTAL SLEEP APPLIANCE

(71) Applicants: Dan Rosen, Calabasas, CA (US); Bryan Keropian, Woodland Hills, CA (US)

(72) Inventors: Dan Rosen, Calabasas, CA (US); Bryan Keropian, Woodland Hills, CA (US)

(21) Appl. No.: 14/188,178

(22) Filed: Feb. 24, 2014

(51) Int. Cl. A61F 5/56

(52) U.S. Cl. CPC A61F 5/566

(57) ABSTRACT

A dental oral appliance for use with patients who suffer with sleep disorders, to reduce or eliminate snoring or obstructive sleep apnea and to open the airway for a sleeping patient. Retention for the appliance is provided by an occlusal coverge, or wire clasps, on the upper or lower teeth. A raised incisor ramp that extends from the incisal tip (biting edge) of the incisors toward the lingual, or posterior ramps, separate the upper and lower teeth to open the airway. A transpalatal bar or translingual bar, which extends from the inside (lingual) of the right molars to the inside of the left molars, inhibits the upward and backward movement of the tongue. A posterior tongue restrainer has two parts, the first part being embedded in the transpalatal bar and the second part removably attachable to the first part, in order to further inhibit the upward and backward movement of the tongue.

### TECHNICAL FIELD

This invention relates to an intra-oral device for reducing or eliminating snoring and/or sleep apnea.

### BACKGROUND

The invention described in this application is an improvement over the devices described in Dr. Keropian's U.S. Pat. No. 6,766,802, issued Jul. 27, 2004, U.S. Pat. No. 7,451,767, issued Nov. 18, 2008, U.S. Pat. No. 7,861,722 issued Jan. 4, 2011, U.S. Pat. No. 7,861,724 issued Jan. 4, 2011 and U.S. Pat. No. 8,132,567 issued Mar. 13, 2012.

As stated in the above listed patents, it has been estimated that ninety million American adults and children snore and that three in every ten adults snores. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death.

Obstructive sleep apnea occurs during sleep when the tongue falls and rolls upward and backward, blocking the airway for 10-90 seconds. These events are measured by spending the night sleeping in a center which measures the number of air blockage events per hour. Less than 5 events per hour is normal. 5-19 events per hour is mild sleep apnea. 20-39 events per hour is moderate sleep apnea. Over 40 events per hour is severe sleep apnea. For sleep apnea there are three main treatments of choice: the CPAP machine, surgery and oral sleep appliances. They are all designed to open the airway during sleep so that there is minimal or no air blockage.

Snoring is caused by vibration of the tissues due to air turbulence as the airway narrows and may be a sign that a patient is suffering from apnea. But not all snorers suffer from apnea. Snoring can be categorized by its severity. There is the snorer who snores but experiences no physical problems. Then, there is the snorer who suffers from apnea, or the snorer who suffers from upper airway resistance. In some of these people, though they may not actually experience apneic episodes, their snoring is so loud and their breathing so labored, that it still wakes them, and their partners, numerous times throughout the night.

Many spouses, partners and/or children suffer through the night from the annoying noise of the snorer. Snoring not only disturbs the sleeping pattern of the snorer himself, it is also disruptive to the family life by causing lack of sleep to all involved. This leaves all involved unrefreshed, tired and sleepy throughout the day. It can cause sleepiness while driving, reading, working or doing other tasks.

A broad variety of inter-oral and dental appliances and devices are now available to treat a patient for snoring. Some known oral devices for treating snoring and obstructive sleep apnea are worn inside of the mouth and work by repositioning of the jaw, moving the mandible, lifting the soft palate or moving the tongue forward. The various classes of treatment devices that now exist include mandibular advancers and tongue advancers. These appliances work by advancing the tongue and soft palate away from the back wall of the throat. Other methods used to treat snoring include controlled positive air-flow pressure systems, also known as CPAP, which require a nose mask, which are quite uncomfortable.

Other treatments for snoring include various surgeries, which are drastic steps to take to attempt to cure the problem, however snoring can be so disruptive to a person's life and relationships, that some sufferers resort to surgery.

### BRIEF DESCRIPTION OF THE INVENTION

The sleep appliance of this invention is a dental oral appliance for use with patients who suffer with sleep disorders. Primarily it is designed to reduce or eliminate snoring and to open the airway for a sleeping individual who suffers with obstructive sleep apnea. Embodiments of the appliance are physically designed similar to an upper (maxillary) or lower (mandibular) bruxism appliance. They cover the inside (lingual) of the upper teeth and have an open palate (nothing covering the middle area of the palate) or the inside (lingual) of the lower teeth. The body of the appliance has a series of recesses to fit against the lingual side of the teeth.

Retention (holding ability) for the appliance is provided by either acrylic fittings which hold the appliance in place in the same manner as an occlusal night guard or wire clasps which attach the appliance to the teeth.

In one embodiment, in the anterior area there is a raised strip or ramp that extends from the incisal tip (biting edge) of two or more of the incisors toward the lingual. It extends back a short distance from the incisors (where they meet or touch each other). This raised anterior strip acts as a bite discluder, to disclude or separate the posterior teeth.

In an additional embodiment there is no anterior ramp. The upper and lower teeth are separated by raised posterior ramps. This embodiment allows more room for the tongue to come forward, if desired.

There is a transverse strip, a transpalatal bar, that extends from the inside (lingual) of the upper or lower right molars to the inside of the upper or lower left molars. This transverse strip extends from the right to the left and covers the tongue. It generally does not touch the tongue unless the tongue attempts to move upward or backward, as often happens during sleep, causing snoring or sleep apnea. The transpalatal bar inhibits and restrains the upward and backward movement of the tongue, keeping the airway open during sleep.

Optionally, the transpalatal bar is slightly curved upward at its center, or it can be straight across, so that it does not touch the tongue but passes just over the tongue when the tongue is in its normal position. Also, the transpalatal bar does not touch the palate. By not touching either the tongue or the palate, the device of this invention is comfortable to wear and easily tolerated by patient users. Prior art devices, which have pushed the palate up, were found to be unusable, as they often were so uncomfortable that they were unable to be tolerated by the user. The exact radius of the curvature of the transpalatal bar is determined by the physical dimensions and structure of each individual patient's anatomy. Some patients may need little or no curvature to achieve optimum results and other patients may need more curvature. The object is to have the transpalatal bar not touch the tongue or the palate, when the tongue is in its normal position, so that it will be tolerated, but inhibit and restrain any upward or backward movement of the tongue during sleep.

In addition, in certain devices of this invention, there is optionally a posterior tongue restrainer (a tail) that extends backward from the center of the transpalatal bar. This posterior tongue restrainer provides a further barrier to the tongue's superior and posterior movement that blocks the airway to the posterior portion of the mouth. The posterior tongue restrainer, like the transpalatal bar, may, or may not, touch the tongue in its normal position and does restrain and inhibit the upward and backward movement of the tongue during sleep. Also, the posterior tongue restrainer, like the transpalatal bar, does not touch the palate. This posterior tongue restrainer may be added to all of the appliances that are described in the above-mentioned issued patents. The need for a posterior tongue restrainer depends on the needs of the patient.

Alternatively, the transpalatal bar may be removed and reattached to another position, using common dental acrylic. With this adjustment, the appliance can be customized to each patient, to provide the proper fit for inhibiting upward and backward movement of the tongue of each patient at its maximum effectiveness.

There can also be a tail piece that extends posterior and vertically downward from the transpalatal bar. It has been discovered that most patients who are treated for snoring and sleep apnea respond favorably, to at least some extent, with at least one of the appliances described above. Some patients do not respond effectively to any of the previous designs. They still have serious snoring and sleep apnea problems. This vertically extending tail is one of the appliances that is designed to press against the tongue to push it down to hold the airway open.

Applicants have discovered in treating hundreds of patients that those patients who have the most serious snoring and sleep apnea problems, who are not sufficiently aided by any of the previously described appliances, are most able to tolerate the vertical tail piece pressing down on the rear portion of their tongue.

The new embodiments described herein are improved appliances that have an extension comprising two parts with means to connect the two parts together. This will be described in detail below. The appliance may be one that fits on the upper or lower teeth. The appliances have a transpalatal bar (upper appliance) or translingual bar (lower appliance) that crosses over the tongue from right to left, as described above. The transpalatal bar or translingual bar may be straight or be curved depending upon the needs of the patient.

To understand the effectiveness of the appliance, the mechanism of snoring and obstructive sleep apnea must be understood. While we sleep, the tongue falls back and up towards the palate and it partially or completely obstructs or closes the airway path. This results in snoring, obstructive sleep apnea, or Upper Airway Resistance Syndrome. The medical treatment for these maladies range from medication to a CPAP (Continuous Positive Airway Pressure) machine. The CPAP is nearly 100% successful when utilized. Unfortunately, the noncompliance for CPAP use ranges from 50% to 80% depending where one searches in the literature. The American Association of Sleep Medicine designated dental sleep appliances as the number one alternative to CPAP for mild and, moderate sleep apnea.

The sleep appliance of this invention is designed to treat the problem of tongue blockage when sleeping. It works by utilizing several factors. First, it changes the vertical dimension (height of the opening or separation of the teeth). This results in an increased opening of the airway. Second, the transpalatal bar and the new two-part extensions act to effectively inhibit and restrain the upward and backward movement of the tongue, which would block the airway opening during sleep.

### OBJECTS OF THE INVENTION

Accordingly, several objects and advantages of the invention are as follows:

It is an object of this invention to provide a simple device to prevent or reduce snoring as well as Obstructive Sleep Apnea.

It is another object of this invention to provide a device, easily applied and easily tolerated, which will substantially prevent snoring.

Further objects and advantages will become apparent from a consideration of the following description and drawings.

### BRIEF DESCRIPTION OF THE DRAWINGS

FIG. 1 is a perspective view, from the bottom of an embodiment of the improved sleep appliance of this invention;

FIG. 2 is a bottom view of an acrylic embodiment as shown in FIG. 1;

FIG. 3 is another bottom view of the embodiment of FIG. 1, using wire clasps;

FIG. 4 is a rear view an embodiment with a straight transpalatal bar;

FIG. 5 is a rear view of an embodiment with a curved transpalatal bar;

FIGS. 6, 7 and 8 are bottom views of second part connectors.

### DETAILED DESCRIPTION

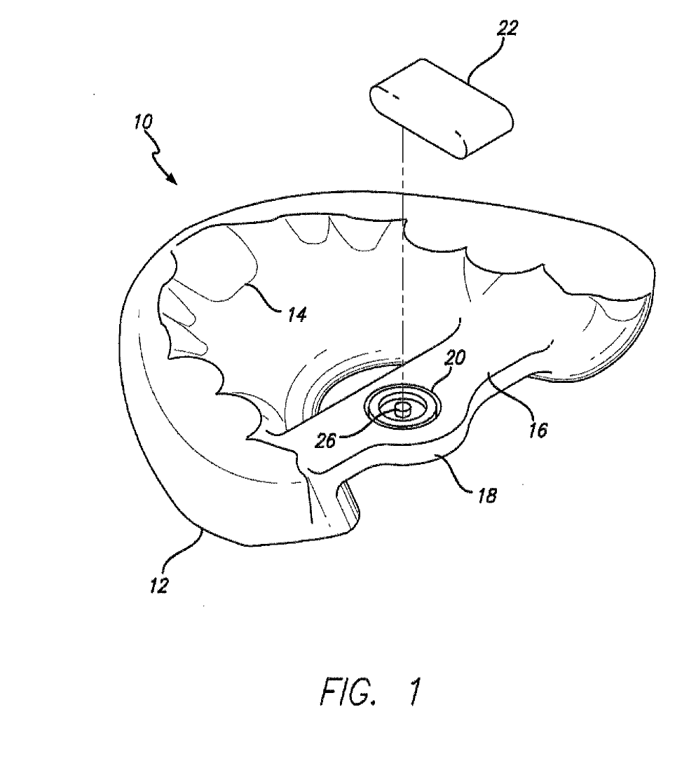

Referring now to FIGS. 1-8, there is shown an embodiment 10 of the dental sleep appliance of this invention comprising a body 12. Body 12 is made entirely of an acrylic plastic, commonly used for dental devices, and is custom made to fit over the wearer's posterior teeth in the same manner as an occlusal night guard, which uses an occlusal coverage. The occlusal coverage holds appliance 10 firmly onto the upper or lower posterior teeth. This posterior occlusal coverage contacts the posterior teeth of the opposing arch and establishes the occlusion for the appliance.

There is a second pattern of occlusion. The occlusion only occurs in the anterior portion of the appliance. There is a raised anterior strip 14 that can extend from about 3 mm anterior of the incisal tip (biting edge) of two or more of the incisors toward the lingual. Strip 14 extends back a short distance from the middle of the central incisors. Strip 14 acts as a bite discluder, separating the posterior teeth. Strip 14 is preferably from about 3 mm to about 5 mm thick in order to separate the posterior teeth.

Transverse transpalatal bar 16 extends from the inside of the right lower molars to the inside of the left lower molars and inhibits the upward and backward movement of the tongue, to keep the airway open during sleep. Transpalatal bar 16 may be straight or curved upwards over the tongue, depending upon the needs of the patient. Transpalatal bar 16 does not touch the tongue in most cases and does not touch the palate. Transpalatal bar 16 does inhibit and restrain the upward and backward movement of the tongue.

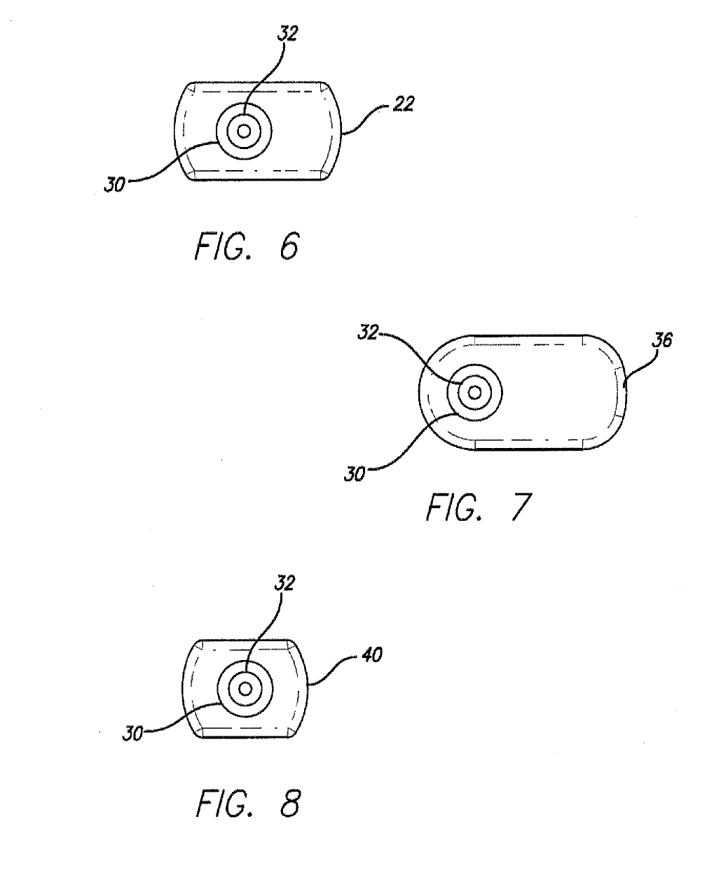

Transpalatal bar 16 has a curved or straight post extension 18 in which one, first part 20 of a two-part coupling is embedded in transpalatal bar 16. The second part 22 of the coupling has means to removably connect to first part 20, shown in FIGS. 6, 7 and 8 and described below. As shown, the connection is by a snap. That is, the second part 22 snaps into first part 20. However, any common type of coupling, such as a snap, can be used, so that second part 22 can be removed (snapped out) and replaced (snapped in) by another part having the same type of snap. Second part 22 acts as a posterior tongue restrainer to inhibit and restrain the upward and backward movement of the tongue.

The purpose of the removable and replaceable coupling part 22 is this. A patient comes in having a snoring or apnea problem. A dental appliance, such as 10, is manufactured to fit into the patient's mouth to wear primarily while sleeping. Part 22 is manufactured in a series of different shaped parts in various sizes. The dentist attaches one of the smaller 22 parts by snapping it into place in part 20. The purpose of the part is to extend down to keep the tongue from moving up in sleep closing the air passage. The patient takes it home and uses it for a few days or up to a few weeks. If the patient advises that they are still having trouble snoring, or has apnea symptoms, he or she goes back to the dentist who replaces a different 22 larger size part to keep the tongue from moving up and backward. The dentist can change the 22 part in a matter of seconds since they each snap into place.

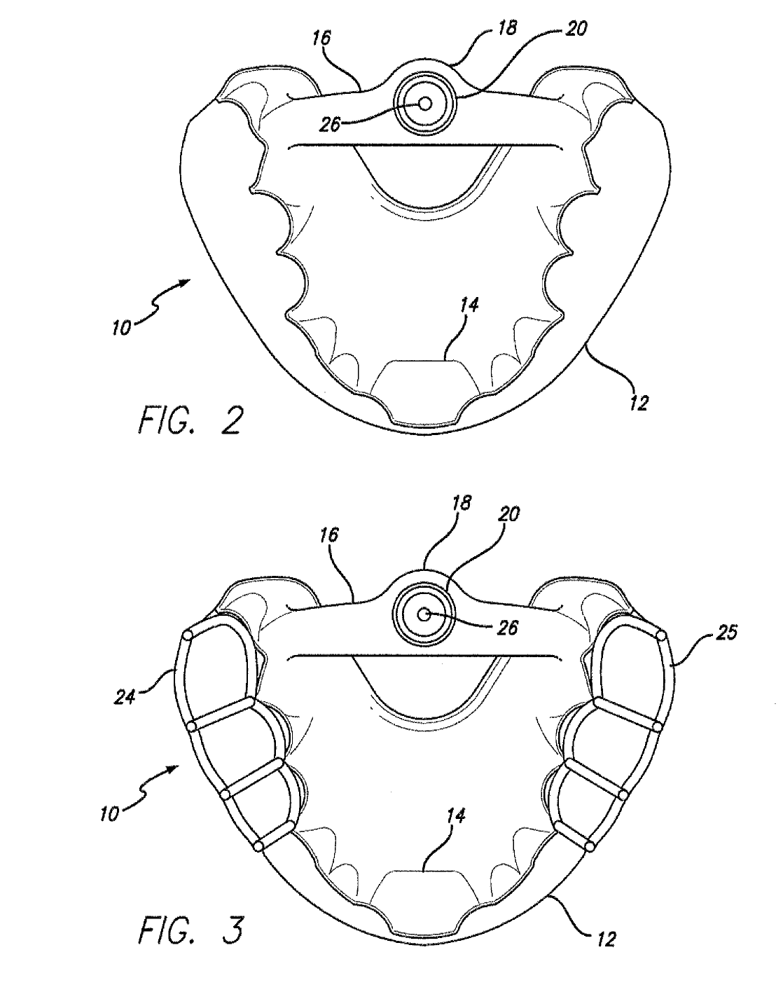

FIG. 2 shows appliance 10 with body 12 made of acrylic, as described previously. FIG. 3 shows the appliance which is made of acrylic but is attached to the rear teeth by a set of wire clasps 24 and 25. The dentist determines which manner of holding the appliance in the mouth is best, depending upon the needs of the patient.

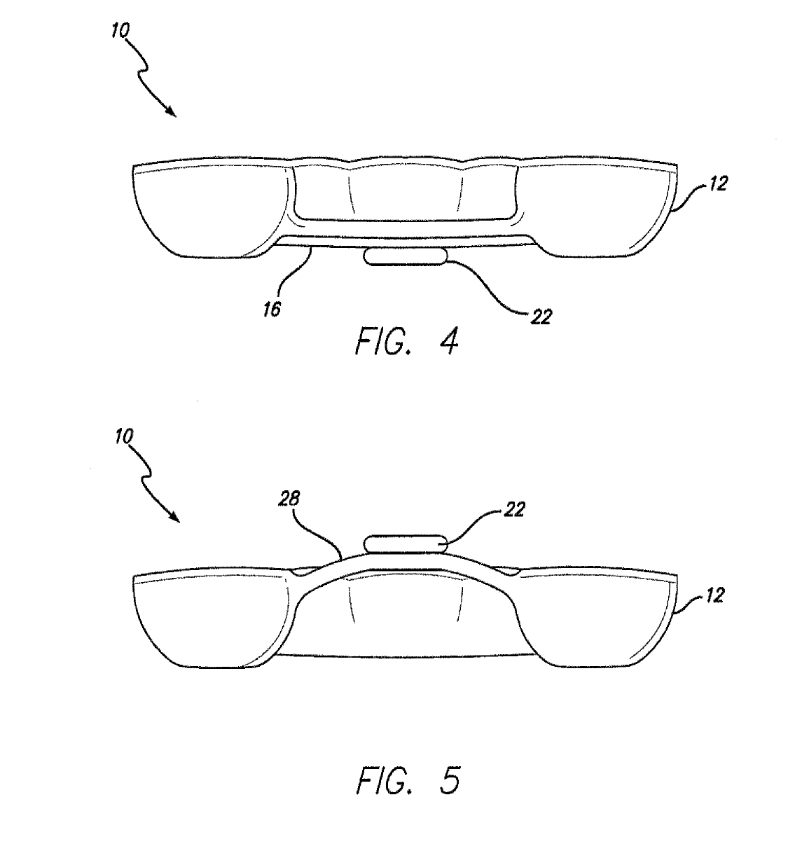

FIG. 4 is a back view of the appliance 10 with body 12 in which the transpalatal bar 16 is straight and part 22 is attached to transpalatal bar 16 by the coupling previously described. FIG. 5 is a back view of the appliance 10 with body 12 with a curved transpalatal bar 28 and with part 22 attached to transpalatal bar 28 by the coupling previously described.

FIG. 6 shows the top side of a posterior tongue restrainer 22 with a second part snap coupling 30. This common type of snap shown in the drawings, shows that first part 20 has a center post 26 while second part 30 has a ring 32 which snaps around post 26 in the first part coupling 20 to lock the two parts together. The two parts can be separated by pulling the two parts apart with reasonable force. It is necessary that the parts when snapped together should be held relatively tightly, so that they do not come apart while the patient is sleeping. Other coupling method examples include use of 2-6 snap-coupling buttons.

FIG. 7 shows another posterior tongue restrainer 36 with the same second part snap 30 with ring 32, where the body of the restrainer 36 is simply larger. FIG. 8 shows a smaller posterior tongue restrainer 40 with the same type of second part snap 30 with ring 32. Second part snap 30 may protrude slightly to connect with first part 20 or the first part 20 may protrude and the second part 30 may lie flush with the bottom of the part, such as 22.

The body of part 22 can be made in any shape and size. A series of many shapes and sizes are manufactured, ready to be used to replace an earlier one tried, until the right size and shape is reached for best results. Various methods of connecting the two parts 20 and 22 together may be used. Snaps, buttons, so long as the part 22 is safely attached so that it does not come loose while the patient is sleeping. Up until today, dentists have had to use the method of adding additional acrylic, which requires the time and effort of melting acrylic and adding it to the appliance. The dentist may not have the time to do it while the patient is in the office, so much time and effort is wasted and patient may have to wait days without an appliance. With a variety of sizes and shapes premade, it takes only seconds to snap out one part and snap in another. In addition, the exact size and shape being used is thus exactly known in advance. In addition, acrylic may be used to attach the tail to the transpalatal bar or translingual bar in addition to the snap-on mechanism.

The length of the posterior tongue restrainer portion 22 can extend from about ¼ inch to about 2½ inches in length from the rear of transpalatal bar 16, and be about ¼ inch to about 1 inch wide, from side to side. Whether the patient needs a short or an elongated posterior tongue restrainer portion must be determined by the medical practitioner trying different models on the patient until it is determined just what model works best in preventing snoring, while at the same time, being comfortable and easily tolerated by the patient. The posterior tongue restrainer 22, may, or may not, touch the tongue, or may press on the tongue in either one spot or the entire length, depending upon the needs of the patient.

As described above with respect to the appliances that fit on the upper teeth, the lower teeth appliances can be fitted with tongue restrainers which curve or are straight, all depending upon the needs of the patient. In addition, the posterior tongue restrainer 22 can be flat in shape, tubular in shape, or any other shape as needed.

On any of the above-described embodiments, it may be necessary to advance the mandible to increase the airway even more and then acrylic is added to the most lingual portion, of the anterior ramp 14, creating a projection wall that comes off of the anterior ramp at 90 degrees. The lower anterior teeth swing forward and bite forward of this lingual wall. This results in the mandible coining forward to obtain an increased opening.

Any of the embodiments described herein may use posterior ramps in place of an anterior strip to disclude the upper and lower teeth. In this device, a base plate is placed over the right and left posterior ramps, anywhere from the 2nd molar to the 1st bicuspid and locked in place with acrylic. In this instance the lower teeth can be advanced forward so that the upper and lower incisors are even, edge-to-edge. Utilizing the posterior ramps, the bite can be in a normal backward placement, centric occlusion, or brought forward so that the incisors are even edge to edge. This is accomplished by adding wet acrylic to the top of the posterior coverage ramps (posterior occlusal surface of the appliance) and having the patient bite into this wet acrylic at the desired anterio-posterior position.

All of the devices described herein, which are totally or partially made of plastic, are preferably made of acrylic plastic or thermal plastic or a combination thereof.

What is claimed is:

1. A dental oral appliance to open the airway for a sleeping individual who suffers with snoring or obstructive sleep apnea comprising, a body portion covering the upper or lower teeth, means to removably affix the appliance to the upper or lower teeth, means to prevent occlusion of the upper and lower teeth, a transpalatal bar that extends from the inside of the right molars to the inside of the left molars to inhibit the upward and backward movement of the tongue, and a posterior tongue restrainer having two parts, the first part being embedded in the transpalatal bar and the second part removably attachable to the first part in order to further inhibit the upward and backward movement of the tongue.

2. The dental oral appliance of claim 1 in which the transpalatal bar is curved.

3. The dental oral appliance of claim 1 in which the first and second part are connected by one of a snap fit, multiple snap fits or acrylic.

4. The dental oral appliance of claim 3 in which the first part is entirely embedded in the transpalatal bar with the exception that the top of the first part is uncovered so that the second part can be attached.

5. The dental oral appliance of claim 1 in which the second part can come in a variety of shapes and sizes.

6. The dental oral appliance of claim 5 in which the second part can be removed and replaced by another second part of a different shape or size, depending upon the need of the patient.

7. The dental oral appliance of claim 1 in which the means to prevent occlusion of the upper and lower teeth comprises a raised incisor ramp that extends from two or more incisors toward the lingual.

8. The dental oral appliance of claim 1 in which the means to prevent occlusion of the upper and lower teeth comprises raised posterior ramps.

9. The dental oral appliance of claim 1 in which the appliance is made of acrylic plastic, thermal plastic or a combination thereof.

10. The dental oral appliance of claim 1 in which neither the posterior tongue restrainer nor the transpalatal bar touches the tongue in its normal state but both inhibit the upward and backward movement of the tongue during sleep.

11. The dental oral appliance of claim 1 in which the posterior tongue restrainer presses down on the tongue.
